(19) United States
(12) Patent Application Publication
(10) Pub. No.: US 2015/0313640 A1
(43) Pub. Date: Nov. 5, 2015

(54) SURGICAL INSTRUMENT WITH MOVABLE GUIDE AND SLEEVE

(71) Applicant: Andres Eduardo O'DALY, Baltimore, MD (US)

(72) Inventor: Andres Eduardo O'DALY, Baltimore, MD (US)

(21) Appl. No.: 14/265,486

(22) Filed: Apr. 30, 2014

(51) Int. Cl. A61B 17/56

(52) U.S. Cl. CPC A61B 17/56

(57) ABSTRACT

A surgical instrument for engaging, aligning and maintaining displaced bone fragments together in a reduced position. The instrument includes first and second lever arms pivotally attached to one another. The arms have a proximal end and a distal end with an engaging tip to grip the fractured bone fragments without interfering with the preparation and placement of the bone fixation device. An arm locking mechanism is configured to hold the engaging tips in a desired position relative to the bone fragments to maintain the fractured bone in a reduced position. An alignment guide is movably attached to one of the lever arms so that it can rotate through multiple axes. A removable guide sleeve is provided for the alignment guide to guide the orientation of drill bits, tap tools, and countersink tools used to prepare the bone fragments for placement of the fixation device in the fractured bone.

### TECHNICAL FIELD

The disclosure is directed to a medical device that is used as a reduction clamp for orthopedic surgery. More particularly, the disclosure is directed to an integrated movable guide, removable guide sleeve and reduction clamp for fracture reduction and placement of lag screws by orthopedic surgeons.

### BACKGROUND AND SUMMARY

Reduction is a medical procedure used to restore a bone fracture to the correct alignment for proper healing. When a bone fractures, the fragments of the bones may lose their alignment either by displacement or by angulation. The surgeon needs to re-align the bone fragments to their normal position so that the fractured bone may heal without deformity.

Reduction clamps, otherwise known as forceps or graspers, may be used by a surgeon to position and hold bone fragments for the treatment of fractures. Treatment may include fixation of the bone fragments to one another through the use of plates, guides, wires, screws, and the like. During an orthopedic surgery procedure, a doctor may use a range of reduction clamps for different size drills in order to drill holes in the bone for inserting fixation screws in the bone. Such reduction clamps are designed to be reused on multiple patients for reduction and fixation of fractures of various bones in the body. Accessories like plates, guides, and fixation screws that work with specific brands of clamps are available through a variety of medical equipment supply companies.

Reduction clamps have a variety of designs. Some reduction clamps have pointed tips while other clamps have balls or serrated gripping surfaces. The clamps have arms that can open to varying widths and typically have a locking mechanism to allow precise control of the opening angle of the arms of the clamps. Once a pair of reduction clamps is in place, the surgeon may engage the locking mechanism to maintain the opening angle of the clamp in a fixed position, which also allows for hands-free use of the clamp.

The reduction clamps may be used to carefully manipulate bone fragments while surgically treating a fracture. The same reduction clamp may also be used to hold the bone fragments in reduced position while the surgeon places a fixation device (e.g screw, place, wire, etc.) through the bone fragments to keep the bone fragments stabilized during the healing process. In a fracture where the bone breaks into two or more segments, a set of forceps may be used to manipulate the bone segments. Surgeons may carefully position the bone segments in order to realign the segments and/or reduce the fracture so the bone segments can be set in place for proper healing. While manipulating and reducing a fracture the tips of the reduction clamps are placed in a perpendicular axis to the fracture line. Once set in place, a fixation device, such as a screw may be inserted through the bone fragments. For proper compression of the bone fragments and healing, the screw must be inserted perpendicular to the fracture line. The optimal position for the compression screw almost always coincides with the axis between the tips of the reduction clamp. Accordingly, it is critical to be able to drill a hole for the screw in the bone fragments at an angle that is precisely perpendicular to the fracture line. Since the screws are typically tapered, the holes in the bone must be drilled with different diameter drill bits making it necessary to use different diameter drill guides. However, when removing and replacing the drill guides for drilling the bone fragments, it is difficult to get the guides positioned in precisely the same location at precisely the same angle.

In view of the foregoing, an embodiment of the disclosure provides a surgical instrument for simultaneously engaging, aligning and maintaining displaced bone fragments of a fractured bone together in a reduced position in order to facilitate preparation and placement of a bone fixation device in an axis perpendicular to a fracture line between the bone fragments. The instrument includes first and second lever arms pivotally attached to one another. Each of the first and second arms have a proximal end having a handle and an arm locking mechanism, and a distal end with an engaging tip configured to grip the fractured bone fragments without interfering with the preparation and placement of the bone fixation device. The arm locking mechanism is configured to hold the engaging tips in a desired position relative to the bone fragments in order to maintain the fractured bone in a reduced position. An alignment guide movably attached to one of the lever arms. The alignment guide is movably mounted on the lever arm for rotation through multiple axes. A removable guide sleeve is provided for the alignment guide to guide the orientation of drill bits, tap tools, countersink tools, depth gage, and the like used to prepare the bone fragments for placement of the fixation device along the axis perpendicular to the fracture line and as close as possible to an axis collinear with an axis through the engaging tips of the lever arms. According to an embodiment of the disclosure, the surgical instrument is specifically adapted for placing lag screws, as the bone fixation device, into the bone fragments.

An advantage of the surgical instrument according to the disclosure is that the instrument contains a movable guide and removable guide sleeve therefor for guiding different diameter drill bits in order to prepare the bone for placement of a bone fixation device or lag/compression screw to fixedly attach the bone fragments to one another.

Another advantage of the surgical instrument according to the disclosure is that the movable guide is attached to a gripping end of the instrument using multi-positional mount that may be used to fix the movable guide along multiple axes in order to enable preparation and fixation of the bone fragments with a fixation device that is collinearly aligned with gripping tips of the instrument along an axis that is perpendicular to the fracture line between the bone fragments.

### BRIEF DESCRIPTION OF THE DRAWINGS

FIG. 1 is a perspective view of a reduction device according to one embodiment of the disclosure.

FIG. 2 is a top plan view of the reduction device of FIG. 1

FIG. 3 is an elevation view of the reduction device of FIG. 1 showing the movable guide is a first angular position.

FIG. 4 is an elevation view of the reduction device of FIG. 1 showing the movable guide is a second angular position.

FIG. 5 is a front elevation view of the reduction device and movable guide of FIG. 1.

FIG. 6 is an exploded side elevation view of a movable guide and portion of a gripping arm of FIG. 1.

FIG. 7 is a perspective view of the portion of the gripping arm for the movable guide of FIG. 6.

FIG. 8 is an exploded view of the guide and removable sleeve therefor for use with the gripping arm of FIG. 7.

FIG. 9 is an exploded side elevation view of a portion of a reduction device, movable guide, and sleeve therefor according to another embodiment of the disclosure.

FIGS. 10A and 10B are schematic illustrations of multiple positions for the movable guide according to the embodiment of FIG. 9.

FIG. 11 is a top plan view of a ball joint device for the movable guide according to the embodiment of FIG. 9.

FIG. 12 is an exploded view of a reduction device according to another embodiment of the disclosure.

FIG. 13 is a perspective view of a movable guide for the reduction device of FIG. 12.

FIGS. 14A and 14B are elevation views of alternative movable guides for the reduction device of FIG. 12.

FIG. 15 is a perspective view of a guide base for the reduction device of FIG. 12.

### DETAILED DESCRIPTION OF THE PREFERRED EMBODIMENTS

The benefits and advantages of the surgical instrument of the disclosure may be further understood by reference to the drawings wherein like numerals indicate like features in the drawings. A surgical instrument 10 is provided for use as a reduction clamp to position and hold reduced bone fragments in the treatment of fractures. For the purposes of this disclosure, bone fractures include single and multiple fracture lines wherein bone fragments are displaced from one another either in an axial direction along a length of the bone fragments or in an angular direction relative to axes aligned along the length of the bone fragments. The instrument 10 includes first and second lever arms 12 that are pivotally attached to one another with pivot pin 14. A proximal end 16 of each of the arms 12 has a handle 18 for manipulating the lever arms 12. A distal end 20 of the lever arms 12 has gripping arms 22 and a guide 24 movably mounted on one of the gripping arms 22 for facilitating the placement of a fractured bone fixation structure in an axis substantially perpendicular to a fracture line between the bone fragments and substantially collinear with an axis through gripping tips 26 of the arms 22. The gripping tips 26 engage bony parts or bone fragments during a surgical procedure to fix the bone fragments in proper alignment to one another and to hold the bone fragments in a reduced position for preparation and placement of a fixation device in the bone fragments. The gripping tips 26 may have one or more sharp edges or points 28 that partially penetrate the bone fragment to provide anti slipping engagement of the instrument 10 with the bone fragments. In other embodiments, the gripping tips 26 may have spherical or serrated gripping surfaces

As shown in FIG. 2, the gripping arms 22 may be arcuate arms 22 wherein the movable guide 24 and gripping tips 26 are offset a distance D from a plane defined by the lever arms 12. The offset distance D will be determined by a degree of angulation of the gripping arms in relation to the lever arm plane, and by the size of the lever arms. This degree of angulation may vary between 0 degrees and 90 degrees to better accommodate the different types of bones and fracture patterns. The offset distance D may range from about 0 to about 50 millimeters relative to the plane defined by the lever arms 12.

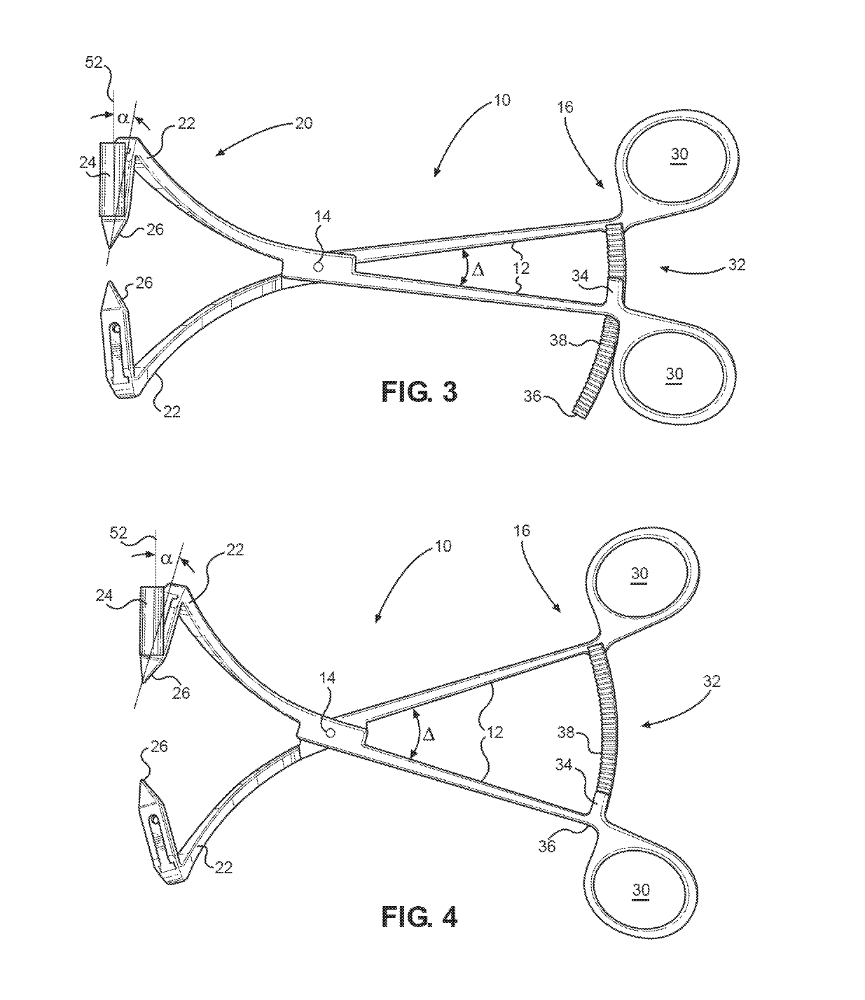

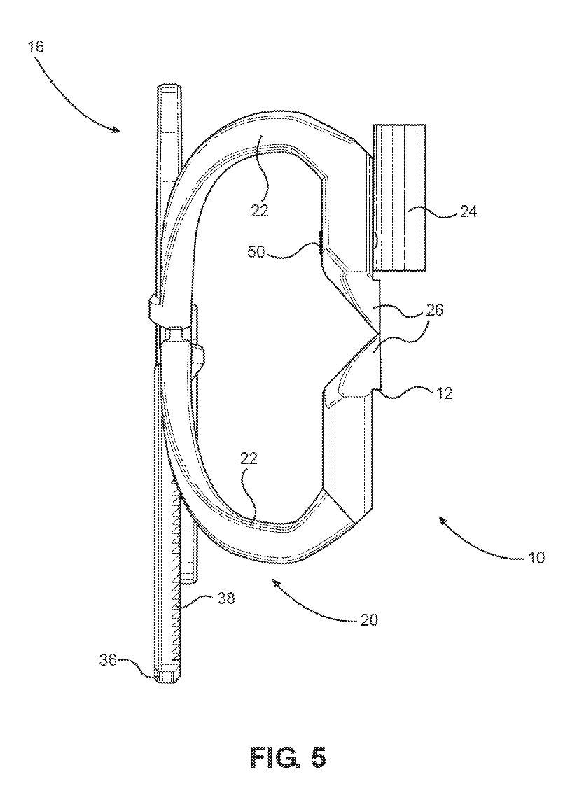

The handle 18 may terminate on the proximal end 16 of the lever arms 12 with one or two loops 30 for finger manipulation of the instrument during the surgical procedure. A locking mechanism 32 may be disposed adjacent the handle 18 of the lever arms 12 for locking the instrument 10 in a predetermined position during the surgical procedure in order to hold the bone fragments in a reduced position. Once locked, the instrument may provide hands free clamping of the bone fragments to one another. The locking mechanism 32 includes a first clamping tab 34 and a second clamping tab 36 each having opposing clamping teeth 38 (FIG. 5) for bias engagement there between. The clamping tabs 34 and 36 enable a fixed angle Δ (FIGS. 3 and 4) to be maintained between the lever arms 12 ranging from about 5 degrees to about 45 degrees, for example, most preferably from about 10 to about 30 degrees.

One of the lever arms 12 has the guide 24 movably attached to one of the gripping arms 22 adjacent the gripping tip 26 thereof. It will be appreciated that the movable guide 24 may be attached to either one of the gripping arms 12. In another embodiment, the guide 24 may include an integral gripping tip for engaging the bone fragments.

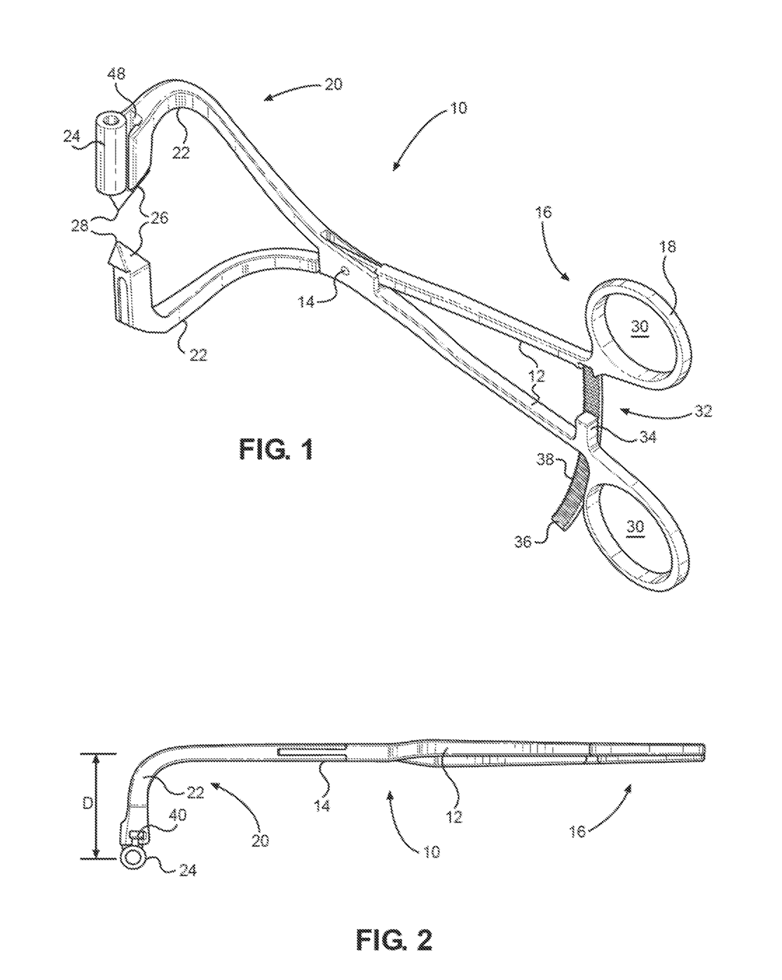

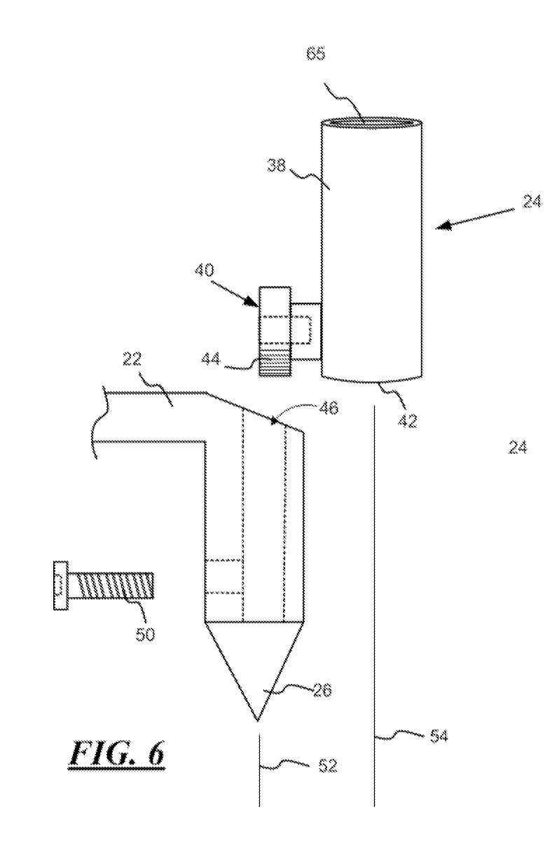

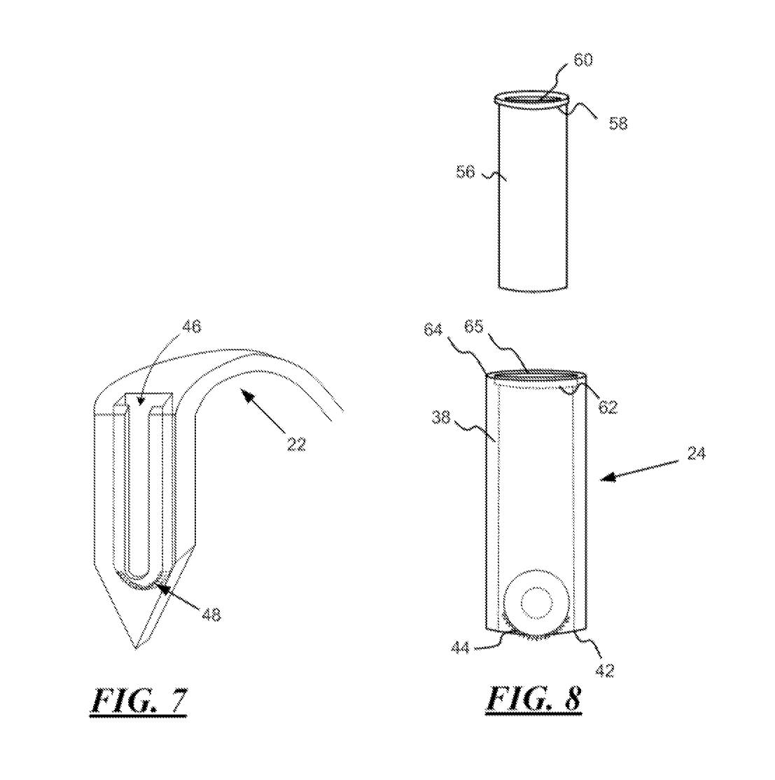

Detailed features of a first embodiment of the movable guide 24 are illustrated in FIGS. 1-6. As shown in FIGS. 3-6, the movable guide 24 is a tubular structure having a multi-axis mount 40 disposed adjacent an end 42 thereof. The multi-axis mount 40 may be provided by a lug having a serrated edge 44. The mount 40 is configured to sliding engagement with a slot 46 in the gripping arm 22. With reference to FIGS. 7 and 8, the slot 46 in the gripping arm 22 may also include serrations 48 for engaging with the serrated edge 44 of the lug 40. A screw or bolt 50 (FIG. 6) may be used to hold the guide 24 in a predetermined position for preparation and fixation of the bone fragments. FIGS. 3 and 4 show the guide in different fixed angular positions on the gripping arm 22 wherein the guide 24 rotates on an axis perpendicular to an axis 52 through the gripping tip 26. As shown in FIG. 5, the lug 40 and slot 46 are configured so that an axis 54 through the guide 24 is closely adjacent to the axis 52 through the gripping tip 26.

As shown in FIGS. 3-4, the slot 46, lug 40, and serrated edges 44 and 48 enable the guide to rotate on the gripping arm 22 to an angle α wherein the guide 24 may then be selectively locked, for example by bolt or screw 50, at the desired angle α that enables a preparation and fixation of the bone fragments with a fixation device that is inserted perpendicular to a fracture line between the bone fragments.

An important feature of the guide 24 is a removable sleeve 56 that is slidably engaged with the guide 24. The sleeve 56 may include a rim 58 for preventing the sleeve from sliding through the guide 24 during placement of the gripping arms 22 on the bone fragments. Multiple sleeves 56 having various diameter openings 60 therein may be used with a single guide 24 to provide the ability to use different diameter drill bits and screws with a single reduction device 10. The sleeve 56 may be inserted and removed from the guide without removing the reduction device from the bone fragments and without a need to readjust the guide relative to the fracture line between the bone fragments. Accordingly, multiple diameter holes may be drilled in the bone more precisely along a single axis wherein minute deviations from the hole axis are minimized or eliminated. The sleeve 56 may also allow placement of a depth gage to measure the length of the drilled holes for adequate placement of bone fixation device/screw.

In another embodiment, the guide 24 may include an internal retaining rim 62 or structure adjacent the end 64 thereof to prevent the sleeve 56 from sliding through the cylindrical opening 65 in the guide 24.

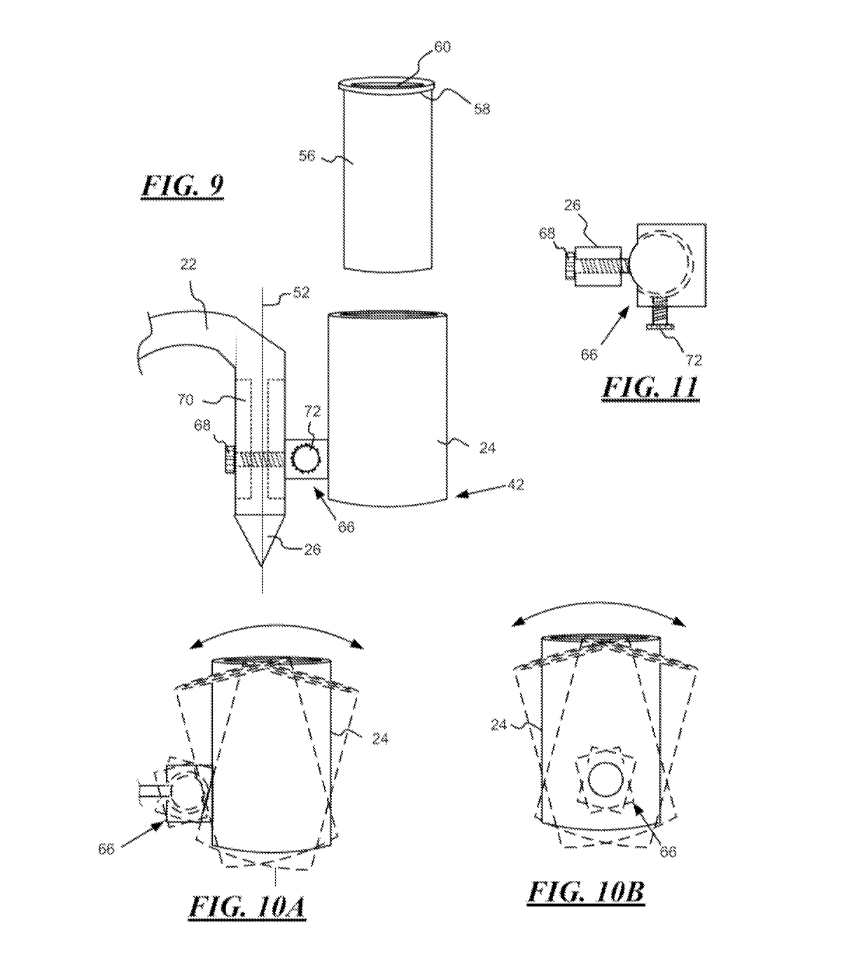

In another embodiment shown in FIGS. 9-11, a mount 66 that includes a ball-and-socket joint or a universal joint may be used to rotate the guide 24 on axis parallel to and/or perpendicular to the axis 52 through the gripping tip 26. Various orientations for the guide 24 are illustrated schematically in FIGS. 10A and 10B. However, with a ball-and-socket joint or universal joint, the axes of rotation of the guide 24 may be substantially unlimited. The mount 66 may be attached to the gripping arm 22 adjacent the engagement tip 26 using an adjustment screw 68 that enables movement and fixation of the mount 66 and guide 24 translationally along the axis 52 in a slot 70 in the gripping arm 22. A second adjustment screw 72 may be attached to the mount 66 to fix the mount 66 and guide 24 in predetermined position that enables drilling or penetration of the bone fragments along an axis that is substantially perpendicular to a fracture line in the bone fragments.

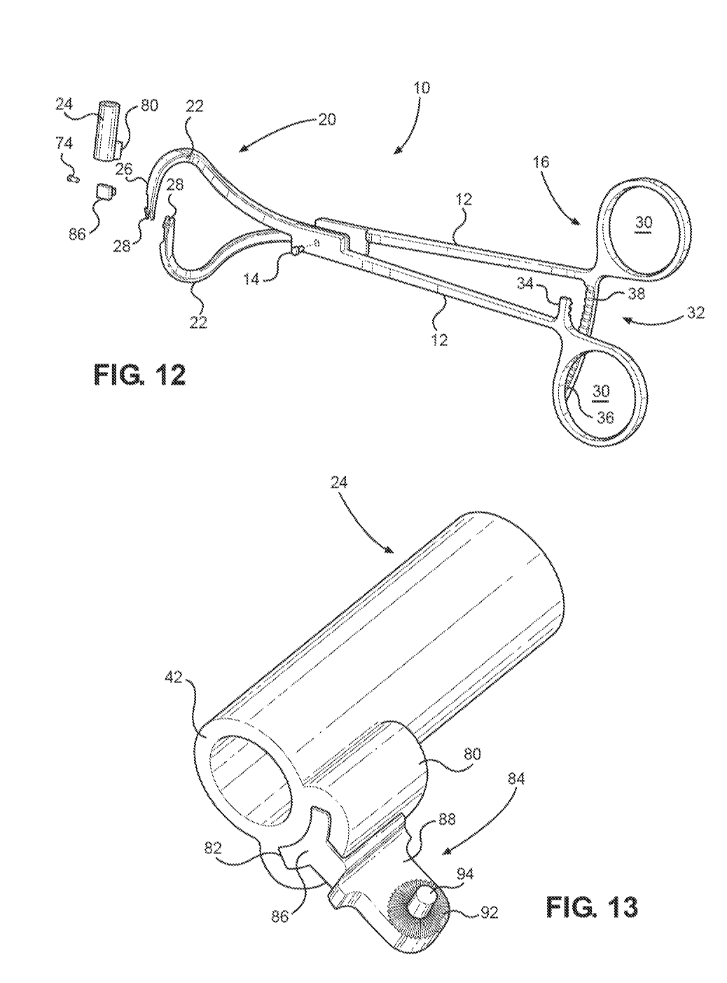

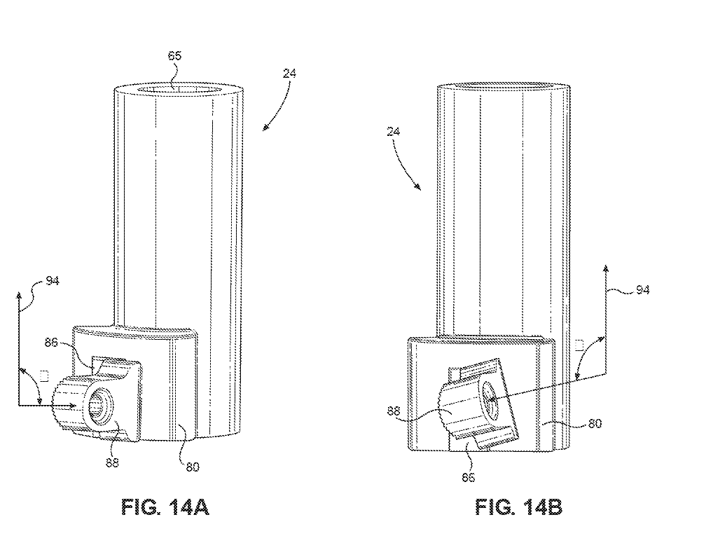

In another embodiment, illustrated in FIGS. 12-15, the guide 24 contains a mounting lug 80 that includes a Y-shaped channel 82 therein for engaging a guide base assembly 84 (FIGS. 12 and 13). The guide base assembly 84 includes a Y-shaped tab 86 for engaging the Y-shaped channel 82 of the lug 80. As in the previously described embodiment, the movable guide 24 enables the use of multiple diameter sleeves 56 with the guide 24 to accommodate different diameter drill bits, tap tools, countersink tools, wires, pins, screws and the like for fixing the bone fragments in a healing position.

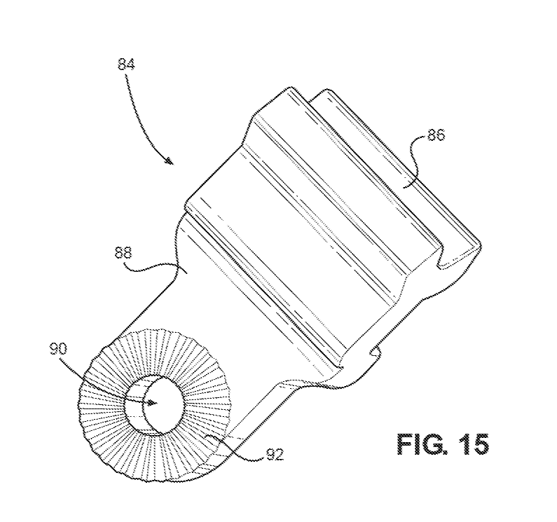

The guide base assembly 84 also includes an aperture arm 88 fixedly attached to the Y-shaped tab 86. The aperture arm 88 has an opening 90 therein (FIG. 15) for pivotally mounting the guide base assembly 84 to one of the gripping arms 22 of the instrument 10. In one embodiment, aperture arm 88 has teeth 92 disposed around the opening 90 for locking engagement with opposing teeth on the gripping arms 22 and for angular rotation of the guide 24 relative to the gripping arms 22. A mounting pin 94 may be disposed in the opening 90 for removably securing the guide base assembly 84 to the gripping arm 22.

In another embodiment illustrated in FIGS. 14A and 14B, the aperture arm 88 of the guide base assembly 84 may be fixedly mounted on the Y-shaped tab 86 at an angle β ranging from about 90 to about 115 degrees relative to longitudinal axis 94 of the movable guide 24.

Accordingly, the disclosed embodiments provide a surgical instrument 10 as a reduction clamp that includes an integrated movable guide and removable sleeve therefor. The reduction clamp and an integrated movable guide and sleeve may be used for simultaneously gripping, aligning and maintaining displaced fragments of a fractured bone together in a reduced position. The clamp may also facilitate the placement of a fractured bone fixation structure in an axis substantially perpendicular to the fracture line and substantially collinear to the axis of the tips of the gripping arms of the reduction clamp.

The previously described embodiments of the present disclosure have many advantages. The foregoing description of preferred embodiments have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention.

What is claimed is:

1. A surgical instrument for simultaneously engaging, aligning and maintaining displaced bone fragments of a fractured bone together in a reduced position in order to facilitate preparation and placement of a bone fixation device in an axis perpendicular to a fracture line between the bone fragments, the instrument comprising:
first and second lever arms pivotally attached to one another, wherein each of the first and second arms have a proximal end having a handle and an arm locking mechanism, and each of the first and second arms have a distal end with an engaging tip configured to grip the fractured bone fragments without interfering with the preparation and placement of the bone fixation device, wherein the arm locking mechanism is configured to hold the engaging tips in a desired position relative to the bone fragments in order to maintain the fractured bone in a reduced position,
an alignment guide movably attached to one of the lever arms, wherein the alignment guide is movably mounted on the lever arm for rotation through multiple axes, and
a removable guide sleeve for the alignment guide to guide the orientation of drill bits, tap tools, countersink tools, and the like used to prepare the bone fragments for placement of the fixation device along the axis perpendicular to the fracture line and as close as possible to an axis parallel with an axis through the engaging tips of the lever arms.

2. The surgical instrument of claim 1, wherein the removable guide is pivotally mounted on the lever arm by a joint configured to allow movement of the removable guide in different planes of rotation and the movable guide includes a locking mechanism for stabilization of the guide in a predetermined fixed position to facilitate the placement of the bone fixation device in the fractured bone.

3. The surgical instrument of claim 1, wherein the removable guide is mounted on the lever arm.

4. The surgical instrument of claim 1, wherein the removable guide further comprises an integral gripping tip for engaging the bone fragments.

5. The surgical instrument of claim 1, further comprising a slot formed in the distal ends of the lever arms for attachment of the movable guide to the lever arm.

6. The surgical instrument of claim 1, wherein the removable guide sleeve includes a rim for preventing the sleeve from moving all the way through the movable guide.

7. The surgical instrument of claim 1, wherein the movable guide includes a retaining rim for preventing the sleeve from moving all the way through the movable guide.

8. The surgical instrument of claim 1, wherein the movable guide is pivotally mounted on the lever arm by a ball joint mounting device configured to allow movement of the movable guide in multiple different planes of rotation, wherein the ball joint mounting devices includes a locking mechanism for locking the movable guide in a predetermined fixed position.

9. The surgical instrument of claim 1, wherein the movable guide is pivotally mounted on the lever arm by a universal joint configured to allow movement of the movable guide in multiple different planes of rotation, wherein the universal joint includes a locking mechanism for locking the movable guide in a predetermined fixed position.
